(19) United States
(12) Patent Application Publication
(10) Pub. No.: US 2024/0374194 A1
(43) Pub. Date: Nov. 14, 2024

(54) Adhesive Wearable Sensors for Measuring Bioelectrical Signals

(71) Applicant: Queen's University at Kingston, Kingston (CA)

(72) Inventors: Shideh Kabiri Ameri Abootorabi, Kingston (CA); Abhijith Balamuraleekrishna Shyam, Mississauga (CA); Anan Zhang, Kingston (CA)

(21) Appl. No.: 18/660,720

(22) Filed: May 10, 2024

Related U.S. Application Data
(60) Provisional application No. 63/465,298, filed on May 10, 2023.

(51) Int. Cl. A61B 5/257; A61B 5/268; A61B 5/279

(52) U.S. Cl. CPC A61B 5/257; A61B 5/268; A61B 5/279

(57) ABSTRACT

A sensor for recording bioelectrical signals directly from hairy skin regardless of the amount or density of hair is fabricated using a polymer with an electrically conductive filler. The sensors have a stemmed conical microstructure array (CMSA) on the sensor surface that interfaces with and adheres to the skin between the hairs. The CMSA sensors are fabricated using a viscosity-controlled dip-pull process (VCDP), including dipping a mold into an electrically conductive polymer precursor having a selected viscosity that is optimized for formation of the conical microstructures upon a controlled pulling of the mold from the polymer precursor.

### RELATED APPLICATION

This application claims the benefit of the filing date of Application No. 63/465,298, filed on May 10, 2023, the contents of which are incorporated herein by reference in their entirety.

### FIELD

The invention relates to the field of sensors for recording electrical signals from the body of subjects. In particular, the invention relates to sensors having microstructures that allow the sensors to adhere to hairy skin.

### BACKGROUND

Early detection of neurological/brain and mental disorders plays a major role in preventing the development of related diseases. However, currently, brain and mental disorders are diagnosed using expensive, bulky, and stationary equipment in hospitals and medical centers by neurologists and skilled clinicians. The lack of accessibility to these services greatly results in the late diagnosis of such disorders. The most commonly used diagnostic tools for brain and mental disease and disorders are magnetoencephalography (MEG) [1], functional magnetic resonance imaging (fMRI) [2], electroencephalography (EEG) [3], and positron emission tomography (PET) [4]. Among these technologies, EEG, which is the non-invasive measurement of the brain's electrical activity through placing electrodes on the scalp, is one of the primary diagnostic methods that can be made portable and integrated with personal and handheld devices for long-term and continuous brain monitoring [5]. EEG is widely used for the diagnosis of epilepsy, stroke, dementia, sleep, cognitive and mental disorders, etc., as it offers a high temporal resolution of milliseconds. The sensors are typically bulky and require a wet gel to provide electrical contact with the skin, and as a result, they are uncomfortable and often unreliable for long-term EEG recording, particularly during daily activities. Further, it is difficult to obtain a reliable signal from hairy skin, which often requires shaving the hair before applying the sensors.

Mobile EEG recording is challenging due to the difficulty of forming a stable interface between sensors/electrodes with the typology of the scalp and the high density of hairs, normally 175 to 300 hairs per square centimeter. The presence of hair negatively impacts the electrode/sensor adhesion to the scalp, electrode-skin interface impedance (ESII), and signal-to-noise ratio (SNR) of the recorded EEG signals. Medical grade EEG recording, for example routine or sleep EEG recording, is usually performed using an array of silver/silver chloride (Ag/AgCl) electrodes held in place using an EEG cap. In order to obtain low ESII and high SNR, a relatively large amount of wet conductive gel (e.g., 0.5 mL) is applied between the scalp and each electrode to ensure a good electrical connection between the electrodes and skin. This placement is time-consuming and it must be done in medical centres and hospitals by trained medical experts based on a 10-20 electrode placement system. Further, the spatial resolution of this test method is low and there is a high chance of shorting between electrodes when they are placed closely in the array due to the gel running and spreading. Therefore, this type of system is not suitable for mobile healthcare.

An alternative recording option is using dry electrodes. Dry electrodes are made of conductive materials that are placed on the scalp and kept in place by mechanical support or the use of aggressive chemical adhesives. The ESII of dry electrodes is comparatively higher than that of wet gel electrodes due to being non-conformal to the skin texture which also results in susceptibility to motion and significant motion artifacts, limiting the application of dry electrodes in mobile health care.

With the advances made in the synthesis of polymers with similar Young's modulus to that of skin, soft polymers coated with conductive films such as metals, nanowires, and graphene have been used to fabricate soft, thin, and stretchable dry electrodes that can conform to the skin's texture resulting in reduced motion artifacts and ESII [6-11]. However, recording EEG from hairy skin using such smooth electrodes is not possible since electrodes do not contact the skin through dense hairs [12, 13].

Recently, use of conductive polymer composites in the fabrication of electrodes and sensors for bio-signal recording has attracted attention due to the low cost, simplicity, and scalability of the fabrication process. The most widely used conductive fillers are metallic or carbon particles, carbon nanotubes (CNT) and nanowires [14-20]. To access the scalp through the hairs, dry electrodes having large pillars on their surface are made of nano and metal particle-based polymer composites. However, such non-adhesive sensors require mechanical support to maintain contact with the scalp. This makes the electrodes/sensors uncomfortable, visually noticeable, and susceptible to motion. Since EEG recordings are greatly affected by motion-induced artifacts, this approach does not provide a reliable solution.

Bio-inspired sensors with micropillars and gecko-inspired hierarchical structures made of nanomaterials-based polymer composites can adhere to hairless skin through Van Der Waal's force, but they are not suitable for application to a hairy scalp due to their planar macrostructures [21-26]. Recently, octopus-inspired or beetle-like self-adhesive sensors based on micro-suction-cup structures made of nanomaterials-based polymer composite have been developed, which again are not able to adhere to a hairy scalp [27,28]. Despite the wide application of polymer composites in bio-signal recording, there is no reliable dry electrode that can conform and adhere to a hairy scalp and provide stable reliable contact without the help of adhesives and/or mechanical support.

### SUMMARY

According to one aspect of the invention there is provided a method for making a stemmed conical head microstructure sensor, comprising: providing a depot of an electrically conductive polymer precursor with a selected viscosity on a substrate; providing a mold comprising a semi-spherical structure for the conical head of the microstructure; placing the mold above the substrate wherein the mold contacts the polymer precursor and a selected portion of the mold is wetted by the polymer precursor; separating the mold and the substrate by a selected distance to allow the polymer precursor to be drawn between the mold and the depot while adhering to the mold according to its selected viscosity, wherein the polymer precursor drawn between the mold and the depot of the polymer precursor forms a stem of the stemmed conical head microstructure sensor; maintaining the selected distance between the mold and the substrate while subjecting the mold and the substrate to a treatment that cures the polymer precursor to form the stemmed conical head microstructure; and removing the conical head from the mold to obtain the stemmed conical head microstructure sensor on the substrate.

According to embodiments, the selected viscosity is about 2000 Pas to about 13500 P a s.

According to embodiments, the selected distance is about 450 μm to about 650 μm.

The polymer precursor may comprise at least one of silicone-based elastomers, polytetrafluoroethylene (PTFE), thermoplastic elastomer (TPE) family of polymers, polyurethane plastics, thermoplastic polyurethane (TPU).

The polymer precursor may comprise a filler selected from graphene, carbon nanotubes, metallic nanoparticles, carbon black, metal flakes, metal nanowire, non-metallic nanoparticles, and combinations thereof.

In one embodiment, the polymer precursor comprises carbon nanotube-polydimethylsiloxane-silicone oil (CNT-PDMS-SO).

One embodiment comprises preparing the CNT-PDMS-SO precursor with the selected viscosity by dispersing CNT in isopropanol alcohol (IPA), SO, and PDMS using ultrasonication, and then heating to remove the IPA to obtain the CNT-PDMS-SO solution.

One embodiment comprises preparing the CNT-PDMS-SO solution with the selected viscosity by mixing about 2.4 wt % CNT and about 20 wt % silicone oil in PDMS, with CNT:SO weight ratio of about 1:8.

The CNT may comprise carbon nanotubes about 10 nm to about 20 nm in diameter and about 10 μm to about 30 μm in length.

In one embodiment the mold comprises one or more glass bead fixed on a face of a second substrate.

In one embodiment the semi-spherical structure has a radius of about 750 μm.

In some embodiments the mold may comprise two or more semi-spherical structures wherein spacing between the semi-spherical structures is about 350 μm to about 450 μm.

The method may comprise separating the mold and the substrate by the selected distance wherein the semi-spherical structure contacts the polymer precursor and about 10% to about 35% of a radius of the semi-spherical structure is wetted by the polymer precursor.

According to another aspect of the invention there is provided a sensor for measuring and/or monitoring one or more bioelectrical signals from the skin of a subject, and/or applying one or more electrical signals to the skin, comprising: a substrate; a stemmed conical head microstructure disposed on the substrate; wherein the stemmed conical head microstructure comprises a polymer composite having a Young's modulus of about 200 kPa to about 1.8 MPa.

In one embodiment the Young's modulus is about 1.7 MPa.

In one embodiment the polymer composite comprises CNT-PDMS-SO.

In one embodiment the polymer composite comprises about 2.3 to about 2.5 wt % of CNT and about 18 to about 22 wt % of SO.

In one embodiment the polymer composite comprises CNT-PDMS-SO with CNT:SO weight ratio of 1:8.

According to another aspect of the invention there is provided a method for measuring and/or monitoring one or more bioelectrical signals from the skin of a subject, and/or applying one or more electrical signals to the skin, comprising adhering a sensor as described herein to the skin of the subject and measuring and/or monitoring the one or more bioelectrical signals and/or applying the one or more electrical signals.

The one or more bioelectrical signal may selected from electromyogram (EMG), electrocardiogram (ECG), electroencephalogram (EEG), electrooculogram (EOG), electroneurogram (ENG), electrochemical skin conductance (ESC), and electrical impedance myography (EIM).

The method may comprise adhering the sensor to the skin regardless of an amount or density of hair coverage on the skin.

### BRIEF DESCRIPTION OF THE DRAWINGS

For a greater understanding of the invention, and to show more clearly how it may be carried into effect, embodiments will be described, by way of example, with reference to the accompanying drawings, wherein:

FIG. 1 is a diagram showing a fabrication process for a conical microstructure array (CMSA) sensor, according to one embodiment.

FIGS. 2A-2F are plots showing electrical and mechanical characteristics of carbon nanotube-polydimethylsiloxane-silicone oil (CNT-PDMS-SO) polymer embodiments, including (A) sheet resistance of polymer samples without SO and with a weight ratio of CNT:SO of 1:8; (B) Young's modulus of polymer samples with different SO:CNT weight ratios; (C) normalized stress-strain curve of polymer with 2.4 wt % CNT and CNT:SO weight ratio of 1:8 versus applied tensile strain; (D) normalized resistance curve of polymer with 2.4 wt % CNT and CNT:SO weight ratio of 1:8 verses applied tensile strain; (E) normalized resistance curve of polymer with 2.4 wt % CNT and CNT:SO weight ratio of 1:8 verses applied compression; (F) polymer with 2.4 wt % CNT and CNT:SO weight ratio of 1:8 under cyclic tensile strain of 20% for 10000 cycles; in FIGS. 2D and 2E, crosses are experimental data and lines are fitted curves.

FIGS. 3A and 3B are diagrams of (A) a conventional medical grade electrode with planar geometry for EEG recording with wet gel applied between electrode and a hairy scalp, and (B) a CMSA sensor with an array of stemmed conical structures that can penetrate through dense hairs which enables direct contact with the scalp and adhesion of the sensor to the hairy scalp for EEG recording, according to a generalized embodiment.

FIGS. 4A and 4B are photographs showing (A) a magnified view of a CMSA sensor with an inset showing a stemmed conical microstructure on the CMSA sensor and (B) a CMSA sensor attached to a hairy scalp, according to embodiments.

FIG. 5 is a plot showing calculated and measured suction force as a function of suction cup radius.

FIGS. 6A-6H are plots comparing electrical performance of a CMSA electrode sensor embodiment and a conventional wet Ag/AgCl electrode, wherein (A) is electrode-skin interface impedance (ESII) normalized to sensor/electrode surface area without any skin preparation; (B) is ECG recording from the chest; (C) is EEG recording in time domain on the forehead with eye open and eye closed; (D) and (E) are power density recordings with eye open and eye closed, respectively; (F) is EEG recording in time domain on a hairy scalp with eye open and eye closed; and (G) and (H) are power density recordings with eye open and eye closed, respectively.

FIGS. 7A-7C show a comparison of motion artifacts of a CMSA sensor embodiment and a conventional Ag/AgCl wet electrode, wherein (A) and (B) are photographs showing placement of the sensor and electrode on a subject's chest and motion induced on the chest using a glass rod, respectively, and (C) shows ECG synchronously recorded by the CMSA sensor and the Ag/AgCl wet electrode.

### DETAILED DESCRIPTION OF EMBODIMENTS

Described herein are conical microstructure array (CMSA) sensors for recording bioelectrical signals directly from skin of a subject, and methods for their fabrication. Embodiments are readily usable on hairy skin, including the scalp, without the need to remove hair from the skin before applying an electrode, as well as on skin without hair. Examples of the bioelectrical signals include, but are not limited to, one or more of electromyogram (EMG), electrocardiogram (ECG), electroencephalogram (EEG), electrooculogram (EOG), electroneurogram (ENG), electrochemical skin conductance (ESC), and electrical impedance myography (EIM). Embodiments may also be used in applications where electrical signals are applied to the skin in, for example, cosmetic, therapeutic, rehabilitation, and training applications, such as transcutaneous electrical nerve stimulation (TENS). Embodiments include an array of stemmed conical microstructures on the electrode surface that interfaces with and adheres to the skin. The conical microstructure array on the surface of the sensor allows the sensor to attach to the skin by contacting the skin between hairs, and thus the sensors are effective regardless of the amount or density of hair coverage, as well as on skin without hair.

The CMSA sensors are fabricated using a cost-effective and time-effective, scalable fabrication process, referred to herein as a viscosity-controlled dip-pull process (VCDP). The fabrication process includes dipping a mold into an electrically conductive polymer precursor having a selected viscosity that is optimized for the formation of the conical microstructures upon a controlled pulling of the mold from the polymer precursor. The mold has one or more semi-spherical structures, typically an array of semi-spherical structures, that provide a cup-like shape of the conical heads of the CMSA. Molds with semi-spherical structures may be fabricated using, e.g., glass beads adhered to a substrate, or techniques such as, but not limited to, 3-D printing and etching. The semi-spherical structures are dipped in the conductive polymer precursor to about 10% to about 35% of their radius, which may be determined according to the size of the semi-spherical structure, and then the mold is pulled away gently, drawing with it polymer precursor on the semi-spherical structures, resulting in elongation of the polymer precursor to form the stems of the conical microstructures. The viscosity of the polymer precursor plays a key role in forming stems which are critical for adhering the CMSA sensor to hairy skin. If the precursor has very low viscosity, a fast capillary rise of polymer within the spaces between the glass beads in the mold will result in failure to achieve proper stemmed structures; if the viscosity is very high, the elongation of precursor and formation of stems will not occur. In embodiments using an array of two or more semi-spherical structures, spacing between the semi-spherical structures may be adjusted to achieve proper capillary rise of the polymer precursor. The spacing may vary according to the size of semi-spherical structures, for example, spacing between semi-spherical structures may be about 350 μm to about 450 μm.

The selected viscosity of the polymer precursor provides for fine adjustment of the capillary rise of the polymer precursor on the semi-spherical structures of the mold without heating so that features such as size and shape of the conical microstructures can be controlled. According to embodiments, the selected viscosity may be about 2000 Pas to about 13500 P a s.

The polymer precursor includes a polymer network or matrix and an electrically conductive filler. The viscosity of the polymer precursor and the mechanical and electrical characteristics of the polymer composite may be controlled and optimized by one or more of the type of polymer, type of filler, size or aspect ratio of filler, and by varying the weight ratio of polymer and filler. Optimization of the viscosity of the polymer precursor within a range of about 2000 Pas to about 13500 Pa s and the conductivity of the polymer composite may be achieved without applying heat or an electric field to the precursor, resulting in a polymer composite with a suitable range of Young's modulus of about 200 kPa to 1.8 MPa.

Examples of polymers include but are not limited to silicone-based elastomers, polytetrafluoroethylene (PTFE), thermoplastic elastomer (TPE) family of polymers, polyurethane plastics, thermoplastic polyurethane (TPU), etc., and combinations thereof. Functionalizing additives with different weight ratio and functionalized fillers or polymer can be also used to alter the mechanical characteristics of the polymer network and thus the polymer composite. Examples of conductive fillers include but are not limited to graphene, carbon nanotubes, metallic nanoparticles, carbon black, metal flakes, metal nanowire, etc., and combinations of such fillers. Consequently, CMSA sensors with different properties of flexibility and conductivity may be prepared.

As used herein, the term “polymer precursor” refers to a polymer with conductive filler that is uncured and ready for use with a mold.

As used herein, the term “polymer composite” refers to a cured polymer with conductive filler in its final form as used in a CMSA sensor.

Owing at least in part to the ease of application of embodiments to hairy skin, and the resulting lack of invasiveness to subjects, they are suitable for a wide range of experimental, research, medical, and technological applications and are suitable for applications requiring long-term continuous use, which may include measuring and/or monitoring one or more bioelectrical signals from the skin and/or applying one or more electrical signals to the skin. For example, embodiments provide a solution for the early diagnosis of brain and mental disorders, and for applications in human-machine interfaces (HMI), artificial intelligence (AI), the internet of things (IoT), assistive technologies, and therapeutic, rehabilitation, and training applications.

As used herein, the term “skin” is intended to refer generally to any part or region of the external surface of an organism from which electrical signals may be obtained or measured, or to which electrical signals may be applied. The term “skin” may include at least the outer layer of the external surface, e.g., the epidermis. The organism may be a plant or an animal, e.g., a human.

As described herein and in the below examples, CMSA sensors (also referred to herein as “electrodes”) form stable physical contact with hairy skin including the scalp and make reliable electrical contact with the skin. Application of a trace amount of conductive gel may be required in some scenarios. Accordingly, CMSA sensor embodiments are suitable for long-term continuous measurements and monitoring in applications such as electroencephalogram (EEG) recording from the hairy scalp and electrocardiogram (ECG) with comparable signal quality to current gold-standard medical grade wet gel electrodes. Although the term “array” is used in general with respect to sensors, it will be appreciated that a sensor having only a single conical microstructure may also be prepared according to the methods described herein.

Embodiments are further described by way of the below non-limiting examples.

### EXAMPLES

### Material and Methods

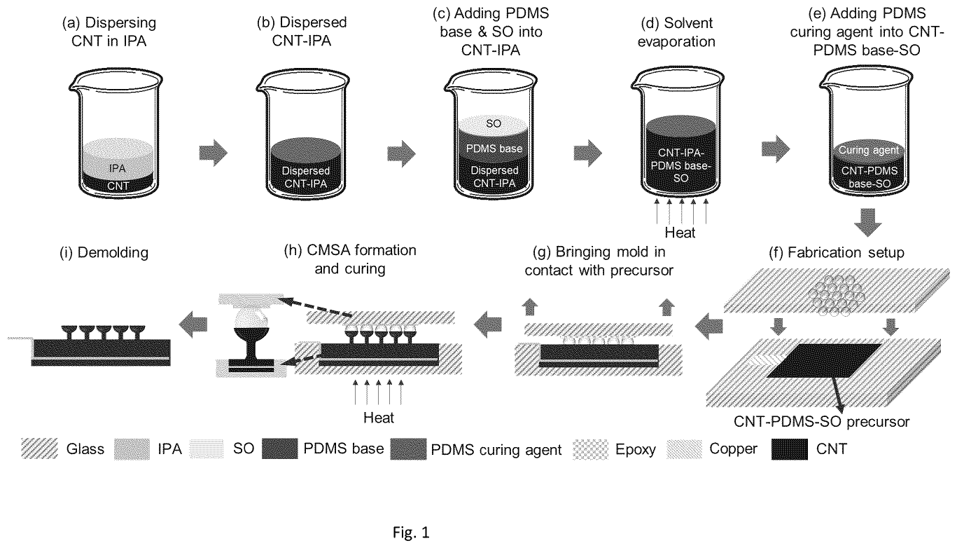

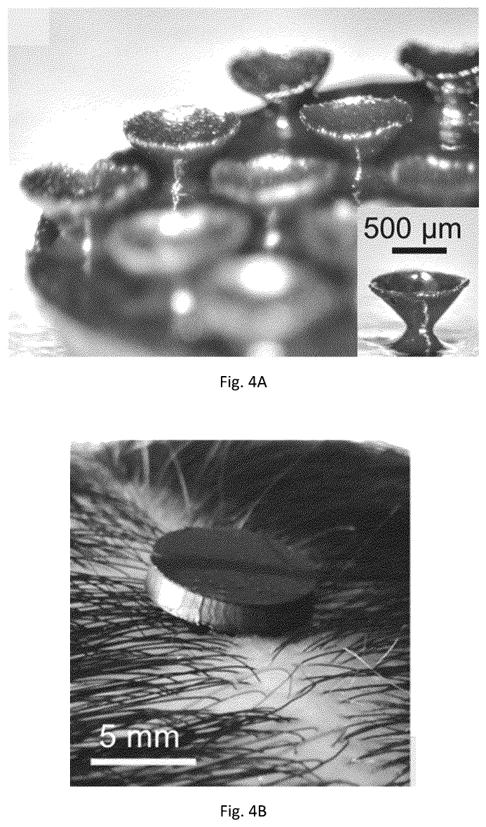

An embodiment of the viscosity-controlled dip-pull process (VCDP) used to fabricate a CMSA sensor is shown schematically in FIG. 1. In general, stemmed conical microstructure arrays were made by dipping a mold into a pool of multiwall carbon nanotube (CNT)-polydimethylsiloxane (PDMS)-silicone oil (SO) precursor with selected viscosity. The mold had an array of semi-spherical structures that provided the cup-like shape of the conical heads of the CMSA (see, e.g., FIG. 4a). To create the mold, an array of glass beads was fixed to a glass slide. The glass beads were dipped to approximately ⅓ of their radius in the precursor polymer to form the conical heads of CMSA, and then the mold was pulled away gently, drawing with it viscous polymer precursor on the glass beads, resulting in elongation of the viscous polymer precursor to form the stems of the conical microstructures. The viscosity of the polymer precursor plays a key role in forming stems which are critical for adhering the CMSA sensor to hairy skin. If the precursor has very low viscosity, a fast capillary rise of polymer within the spaces between the glass beads in the mold will result in failure to achieve proper stemmed structures; if the viscosity is very high, the elongation of precursor and formation of stems will not occur.

In one embodiment the mold was made of an array of glass beads with a diameter of 1.5 mm and spacing of about 350 μm between the glass beads. Approximately 7.4% of the total volume of a glass bead was dipped in the precursor in order to obtain conical microstructures with a larger span-to-volume ratio. A larger span-to-volume ratio results in easier demolding and minimizes the elastic strain on conical microstructures when they are pressed against a contact surface. Other sizes of glass beads may of course be used to create different sizes of conical microstructures.

In some embodiments, CMSA sensors may be made of a conductive polymer composite based on multiwall CNT as conductive fillers and PDMS as the polymer matrix. A fabrication process may start with dispersing CNT in isopropanol alcohol (IPA). CNTs tend to aggregate in polymers and most chemical solvents due to van der Waals forces. In order to obtain a polymer composite with high electrical conductivity, CNTs should be well dispersed in the polymer matrix. This was confirmed in experiments, wherein a CNT-IPA dispersion did not show any visible sign of sedimentation three days after dispersion.

Referring to the embodiment of FIG. 1, In the first step of fabrication (a), each milligram of CNT is dispersed in 0.5 ml IPA using an ultrasonic bath for 60 minutes to form a well-dispersed CNT-IPA mixture (FIG. 1(b). Next, low-viscosity (100 cSt), methyl group terminated (MEP) silicone oil (SO) is added to CNT/IPA dispersion and ultrasonicated for 30 minutes, followed by adding PDMS base and an additional 30 minutes of ultrasonication (FIG. 1c). Methyl-terminated silicone oil coats the CNT surfaces, which brings a more homogeneous dispersion of CNT in PDMS after IPA is evaporated. The silicone oil interacts with the hydrophobic surface of the CNTs to create a thermodynamically stable CNT dispersion in PDMS.

The concentration of silicone oil and the weight percentage (wt %) of CNT are carefully selected to obtain good electrical conductivity as well as optimum viscosity that suits the VCDP fabrication process. In order to obtain the optimal viscosity of the polymer precursor for fabrication of a CMSA sensor, the capillary rise of PDMS precursors with different viscosities was studied. Results suggested that viscosity over about 2000 Pas and below about 13500 Pa s is required to fabricate proper microstructures using the VCDP fabrication process without preheating the precursor.

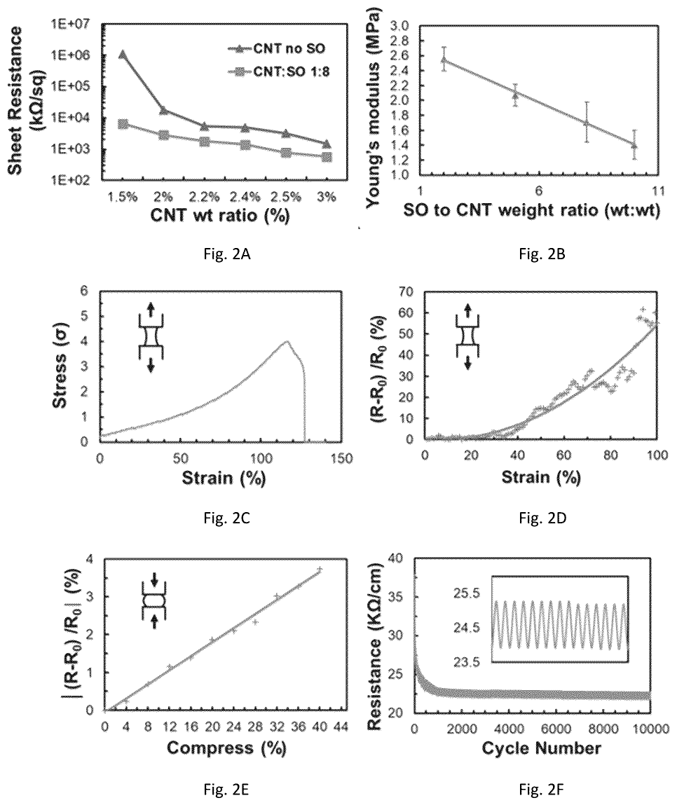

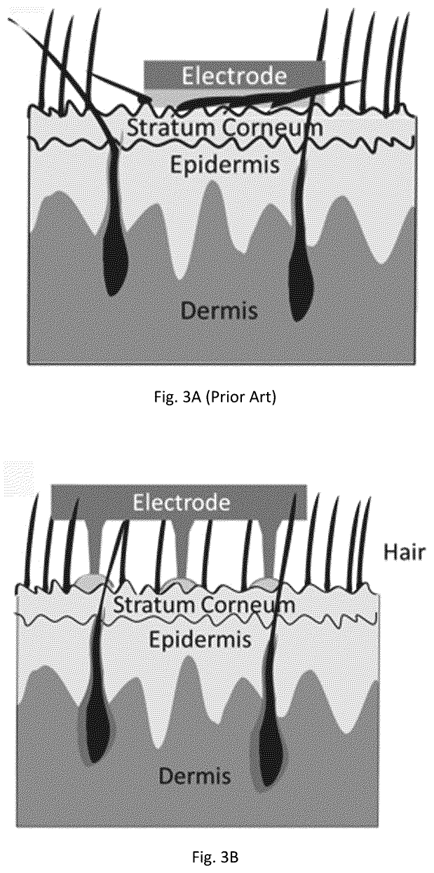

In some embodiments, a CNT-PDMS-SO precursor with proper viscosity and conductivity may be obtained by mixing 2.4 wt % CNT (e.g., multi-wall carbon nanotubes with diameter of about 10 nm to about 20 nm and length of about 10 μm to about 30 μm) and silicone oil (Sigma-Aldrich, 63148) in PDMS (Sylgard™ 184) with CNT:SO weight ratio of about 1:8. A CNT-PDMS-SO precursor with 2.4 wt % CNT was tested with a viscosity of about 12654 Pa·s after adding curing agent. FIGS. 2A and 2B show that electrical conductivity and Young's modulus of the polymer increase by increasing the weight ratio of CNT. The addition of SO to the precursor improves the conductivity and reduces the Young's modulus of the polymer composite. In one embodiment it was found that a polymer composite with 2.4 wt % of CNT and CNT:SO weight ratio of 1:8 had a sheet resistance of 1.387 KΩ/sq and proper mechanical softness for making a CMSA sensor. The CNT:SO weight ratio may be tuned to achieve desired electrical and mechanical properties of the final product for a given application. Generally, if the ratio is decreased the polymer is softer and the electrical conductivity is lower, and if the ratio is increased the polymer is stiffer and the electrical conductivity is higher.

Referring again to FIG. 1, after preparation of CNT-PDMS-base-SO, the mixture was heated at 50° C. to evaporate IPA solvent (FIG. 1(d)). The temperature was kept below the boiling point of IPA to prevent the formation of microbubbles which could degrade the electrical and mechanical properties of the composite. In the next step, a PDMS curing agent with a 1:10 weight ratio of the PDMS base was added to the mixture (FIG. 1(e)). The CNT-PDMS-SO precursor was then placed in a vacuum chamber for de-bubbling.

In one embodiment the fabrication setup includes two parallel stages, one is a fixed stage with a reservoir for polymer precursor equipped with a controllable heating system and a microscope camera to monitor the process. The other stage is movable in x-y-z directions with 2 μm resolution in the z-direction, which acts as a mold holder (FIG. 1(f)). To form conical microstructure arrays, a mold may be made using an array of glass beads of suitable diameters (e.g., 1.5 mm diameter borosilicate glass beads) secured on a glass slide by epoxy glue. A releasing agent (Ease Release™ 200, MANN Release Technologies, Inc.) may be sprayed over the mold to ease demolding of the microstructures. The mold which is initially spaced from the precursor reservoir is lowered in the z-direction and dipped into the CNT-PDMS-SO precursor to form conical heads (FIG. 1(g)). Once glass beads are dipped to approximately ⅓ of their radius in the precursor, the mold is raised up gently which draws up the CNT-PDMS-SO precursor to form pillars (i.e., the stems of the microstructures) with about 450 to 650 μm height and 450 μm to 650 μm diameter (FIG. 1(h)) and held in that position during polymerization. The height of pillars may be adjusted to a lower or higher length depending on the application. In the next step, the precursor is heated at 100° C. for 4 hours to cure the polymer precursor (FIG. 1(h)). Finally, when the CNT-PDMS-SO is cured, the CMSA sensor is demolded (FIG. 1(i)).

### Results and Discussion

Electrical and mechanical characteristics of representative embodiments of fabricated CMSA sensors were measured, and their performance was evaluated.

The Young's modulus of CNT-PDMS-SO composite increases with an increasing weight percentage of CNT, and functionalization with low viscosity silicone oil reduces the elastic modulus of the polymer composite (FIG. 2B).

There is a trade-off between the electrical conductivity and mechanical softness of polymer composite as increasing the weight percentage of CNT reduces the sheet resistance but increases Young's modulus. The electrical and mechanical characteristics of the CNT-PDMS-SO polymer composite with different weight percentages of CNT between 1.5-3%, with no SO added, and with CNT:SO weight ratio of 1:8 were studied. FIG. 2A shows that sheet resistance of polymer composite decreases by increasing the weight percentage of CNT after reaching the percolation at 2 wt % of CNT, and the Young's modulus increases by increasing the CNT weight percentage. Since the addition of SO improves the dispersion of the CNT in the polymer matrix, the CNT-PDMS-SO polymer exhibits lower sheet resistance compared to the polymer without SO. In one embodiment it was found that sufficient electrical conductivity and proper mechanical softness of the polymer composite were obtained at about 2.4 wt % of CNT with a CNT:SO weight ratio of about 1:8. These values may be varied somewhat to adjust the electrical conductivity and mechanical softness of the polymer as desired.

Mechanical characterization of this CNT-PDMS-SO polymer sample indicated Young's modulus of 1.717 MPa (FIG. 2B). The stress-strain curve of the sample, FIG. 2C, shows elastic behavior, with a linear elastic region between 0 to 50% of strain, which was used to extract the Young's modulus of the polymer sample. The fracture strain of the CNT-PDMS-SO sample was measured to be at 116%.

FIG. 2D shows the change in the electrical resistance of sample polymer composites with 2.4 wt % CNT and CNT:SO weight ratio of 1:8, due to applied uniaxial tensile strain. Less than 5% of resistance change was detected within 40% of the applied strain which is well beyond the stretchability of human skin (˜30%) [8]. Additionally, a compression test on the CNT-PDMS-SO polymer embodiment (2.4% CNT, CNT:SO of 1:8) was performed indicating less than 4% resistance change due to 40% of applied compression (FIG. 2E). The normalized change in electrical resistance of this polymer composite sample under the applied cyclic tensile strain of 20% (horizontal to the length of the polymer composite) was demonstrated for 10,000 cycles, shown in FIG. 2F. Over the first 100 cycles an irreversible decrease of electrical resistance was observed, which eventually stabilized after approximately 1,000 cycles of tensile strain. This was attributed to initial changes in the interconnected network of CNTs in the polymer matrix. After further strain cycles there is less breaking and reforming of the CNT networks in the polymer matrix and the resistance stabilizes. Based on these results it was determined that a suitable range of Young's modulus for a CMSA sensor is about 200 kPa to 1.8 MPa

FIGS. 3A and 3B are diagrams showing a conventional planar wet gel electrode and a CMSA sensor embodiment, respectively, on a hairy scalp. As shown in the diagram, hairs lie between the conventional wet gel electrode and the scalp, which prevents the conventional wet gel electrode from making direct contact with the skin. Therefore, the application of relatively a large amount of gel (˜ 0.5 ml) between the electrode and skin is necessary for establishing electrical contact. This results in limited spatial resolution of such electrodes, the necessity of using mechanical support to keep the electrodes in place, a high chance of electrodes shorting when in an array due to the gel running, and the unsuitability of using the conventional electrodes to perform mobile recordings during daily activities. In contrast, the CMSA sensor has stemmed conical microstructures that penetrate through the dense hair and enable direct contact and adhesion of the sensor to hairy scalp skin. A CMSA sensor as described herein may be fabricated in a range of shapes (e.g., circular, elliptical, rectangular, etc.) and sizes as may be convenient for various types of measurements and applications. For example, a circular sensor with a diameter of about 8 mm allows for non-intrusive application for EEG recording from the scalp.

The stems of the conical microstructures make it possible to locate the conical heads in the space between hair strands on the scalp and hairy skin. By gently pressing the CMSA sensor against the skin and pushing the air out of conical heads, a negative pressure is generated which causes the CMSA sensor to adhere to the skin. A trace amount of conductive gel (e.g., about 5 μL) may be applied to the inner surfaces of the conical heads prior to each application to ensure low ESII. As is shown in the diagram of FIG. 3B and the photograph of FIG. 4A, each CMSA sensor has a base where the array of conical microstructures is located and from which they project. Unlike conventional planar wet gel electrodes, CMSA sensors require only a very small amount of gel, making them easy to be attached to the scalp and comfortable to wear. Additionally, they can adhere to a hairy scalp without the help of mechanical support and can be visually imperceptible due to their miniature size and the fact that there is no need for extra accessories to hold them in place, as shown in the photograph of FIG. 4B.

The sensor may have a base with any size depending on the application and a thickness of about 1 mm to 2 mm. The image of the CMSA sensor embodiment of FIG. 4A shows a sensor with 8 mm diameter and 1.5 mm thickness of the base. Each microstructure may have a stem with a radius of about 250 μm±20 μm and a height of about 300±20 μm, and a conical head with a radius of about 450 μm±20 μm, although other sizes may also be fabricated. The number of conical microstructures in the array may be as many as required based on the application. The number may be related to a specific application, where fewer or more conical microstructures would be required or advantageous. For example, when a smaller size sensor is preferred, lower numbers of conical microstructures are provided while for larger sensors a larger number of conical microstructures are provided.

The conical heads of the microstructures are soft (i.e., flexible and resilient) and enable adhesion of the CMSA sensor to both smooth and textured surfaces such as glass and skin respectively. For example, by pressing a stemmed conical head against the skin a negative pressure in the conical microstructure is generated that results in its adhesion to the skin. Application of a CMSA sensor to the hairy scalp is easy and once it is placed on the scalp stays in place and in contact with the skin even during intense head movements. In experimental use, subjects having the CMSA sensors on their scalps reported little to no discomfort, and the sensors are almost visually imperceptible when placed on the scalp, owing to their small size. This would offer privacy to the subjects, especially when long-term EEG recording during daily activities is required. Adhesion forces of a CMSA sensor having of an array of 19 conical microstructures to a glass surface with and without the application of wet gel were measured to be about 201 mN (0.4 N/cm²) and about 103 mN (0.2 N/cm²) respectively.

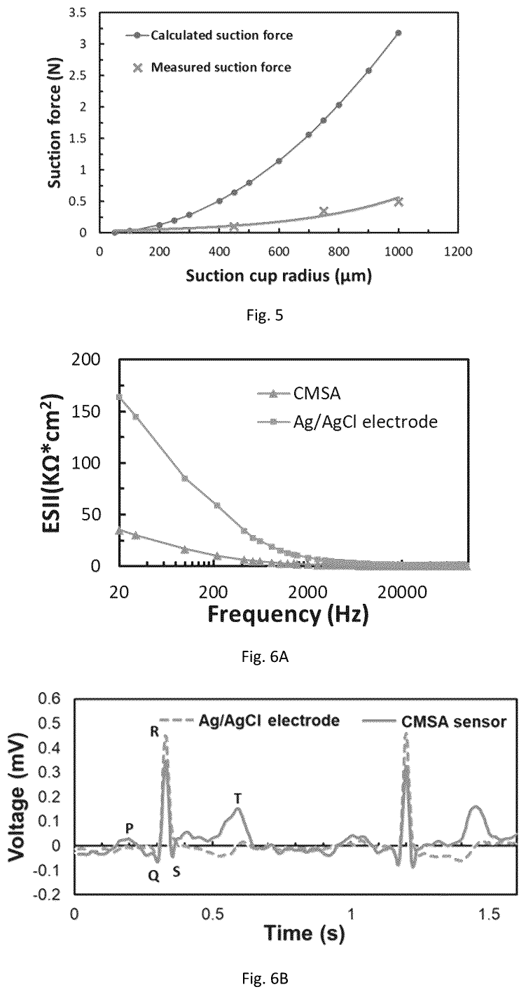

Increasing the radius of the conical heads of a CMSA sensor increases the adhesion force. The adhesion strength of the CMSA sensor is proportional to the size of the conical heads and the negative pressure generated in the conical heads when the air is pressed out. Based on multiple tests of suction forces of conical heads with different radius, the suction force of sensors having 19 conical microstructures with conical heads of different radius was calculated (without gel). The results are shown in FIG. 5 where significant differences between the measured and calculated values are attributed to one or more of deviations of suction cups from their ideal shape and form, slight nonuniformity of the radius of the cups, and the leakage of air into the cups due to conformability of the cups to the attaching surface. There is a trade-off between the sensor's adhesion force and the radius of conical heads since the microstructures must be small enough to fit within the spacing between hair follicles (500 μm to 1 mm in most of the human scalp). CMSA sensors may, of course, be used on skin without hair or substantially without hair, in which case this tradeoff would not be a consideration in selecting the size of the conical heads.

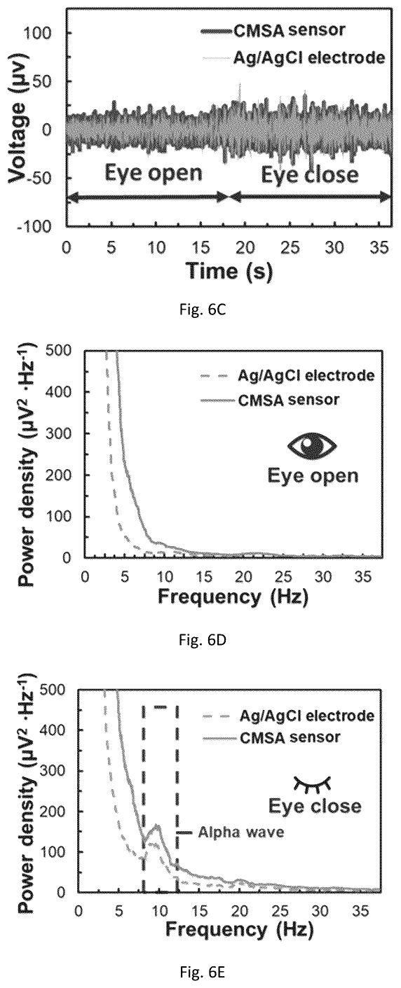

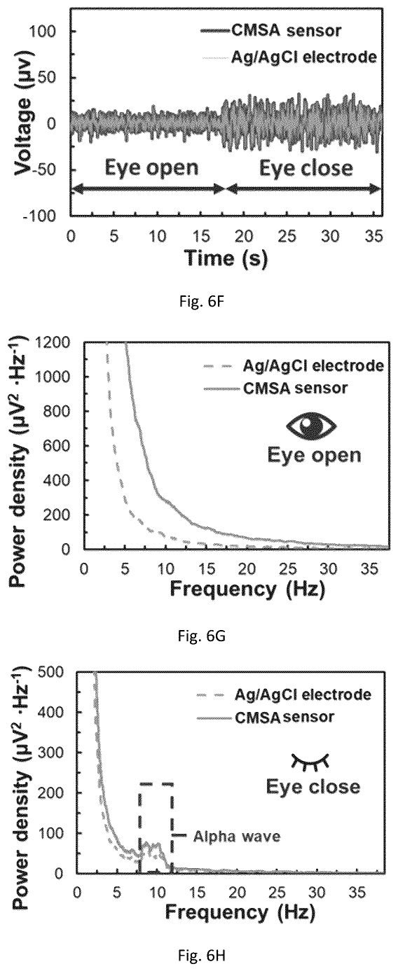

A defining factor for obtaining high SNR and high-quality bio-signal recording is low ESII. The ESII of a CMSA sensor with 19 conical microstructures as in FIG. 4A was measured and compared with a gold standard medical grade Ag/AgCl wet gel electrode ESII. Impedance measurements were performed by placing the electrodes on the forearm and connecting them to an LCR meter, using a sweep frequency of 20 Hz to 100 kHz. The results shown in FIG. 6A suggest lower ESII of the Ag/AgCl wet gel electrode at the interface with skin relative to the CMSA sensor, however, according to electrical circuit theory the ESII is inversely proportional to the contact surface area of sensor/electrodes with skin. Considering that the contact surface area of the CMSA sensor with skin is only about 0.121 cm² and that of the wet gel electrode is about 2.54 cm² (21 times of that in the CMSA sensor), the ESII per unit surface area of the CMSA sensor is lower than the wet gel electrode. The ESII decreases with increasing the frequency which indicates the capacitive nature of the interface between the sensor/electrode and the skin.

The electrical connection between the CMSA sensors and the circuit part is made by embedding copper strips (i.e., conductors) within the polymer composite matrix. The copper strips made strong bonding to the polymer composite. In order to investigate the strength of this bonding, a test sample was prepared that had the same thickness and dimension of the copper strip and the same thickness of polymer composite as in a CMSA sensor, and the force required to pull out the copper strip was measured. It was found that the required force was greater than 16 N. The force required to pull out the copper strip connection is greater than the polymer composite endurance, so that the polymer composite would fail while the copper strip connection remains tightly bonded in the polymer composite matrix.

To investigate the performance of CMSA sensors, EEG and ECG signals were recorded for an embodiment with 19 conical microstructures, as shown in FIG. 4A. A medical grade Ag/AgCl wet gel electrode (from 3M Canada) was also used for comparison. Both electrodes were placed about 3 cm apart on a subject's chest. A trace amount of wet gel was applied onto the inner surface of the CMSA sensor conical heads (about 5 μL) prior to placing on the chest. An OpenBCI board was used for data acquisition and a digital 60 Hz notch filter was applied to the recorded data using MATLAB software. The simultaneously recorded ECG signals using the CMSA sensor and the Ag/AgCl wet electrode are shown in FIG. 6B. The ECG signals recorded using the CMSA sensor and Ag/AgCl electrodes include the signature ECG peaks of P,Q,R,S,T. The average SNR of the signals recorded using the CMSA sensor and wet gel Ag/AgCl electrodes were measured to be 13.74±0.2 and 14.03±0.2 respectively. The ECG signal recording continued for 6 hours after placement of the sensors and the results suggest a comparable average decline of 15.8% and 18.1% in the SNR of the signals using Ag/AgCl electrode and CMSA sensor respectively due to gradual drying out of the wet gel.

Overall it was found that the CMSA sensor shows very good performance for electrophysiological recordings. Although the performance of the Ag/AgCl electrode was slightly superior to the CMSA sensor in some respects, that would be out weighted by the many advantages of the CMSA sensor, including smaller size, higher spatial resolution, superior ability to adhere to hairy skin, ease and comfort of wearing, no need for hair removal, reusability, and visual imperceptibility.

A CMSA sensor and Ag/AgCl wet gel electrode as above were used for EEG recording from the forehead and the scalp of a subject. The alpha rhythm (8 to 13 Hz) was measured using the CMSA sensor and the Ag/AgCl wet gel electrode simultaneously from both the forehead and hairy scalp at F4 position based on 10-20 EEG measurement system. Reference and ground electrodes were placed on the A1 position and wrist [29, 30]. To perform the alpha rhythm recording, the subject was asked to hold their eyes open for 30 seconds followed by another 30 seconds when they were asked to close their eyes and relax. The recorded EEG signals in the time domain are shown in FIGS. 6C and 6F for the forehead and scalp, respectively. The results confirm the CMSA sensor can be used to record EEG signals from both the forehead and hairy scalp. The corresponding fast Fourier transform (FFT) results of the recorded signals when the eyes were opened and closed are shown in FIGS. 6D and 6E (forehead) and FIGS. 6G and 6H (hairy scalp). The results show clear alpha signals recorded using both the CMSA sensor and Ag/AgCl wet gel electrodes at a peak frequency of about 10 Hz during the period at which the eyes were closed.

The long-term continuous EEG recording capability and the adherence of the CMSA sensor from/to the hairy scalp were tested by attaching it to the hairy scalp (O1 and A1 positions) without the help of external mechanical support or tape while the EEG signal was measured continuously for 6 hours. The 6 hours of continuous EEG recording included 120 sets of Alpha signal acquisition. In each set, the subject was asked to close the eyes for one and a half minutes and then open the eyes for another one and a half minutes. The first observable degradation in the signal quality was seen after 4 hours due to the separation of some of the conical heads from the skin. Signal quality was restored by gently pressing the sensor against the skin to re-establish the contact between separated conical heads and skin. The recording then continued for another 2 hours. The CMSA sensor can be reused by rinsing it with water and re-applying the conductive gel before the next use.

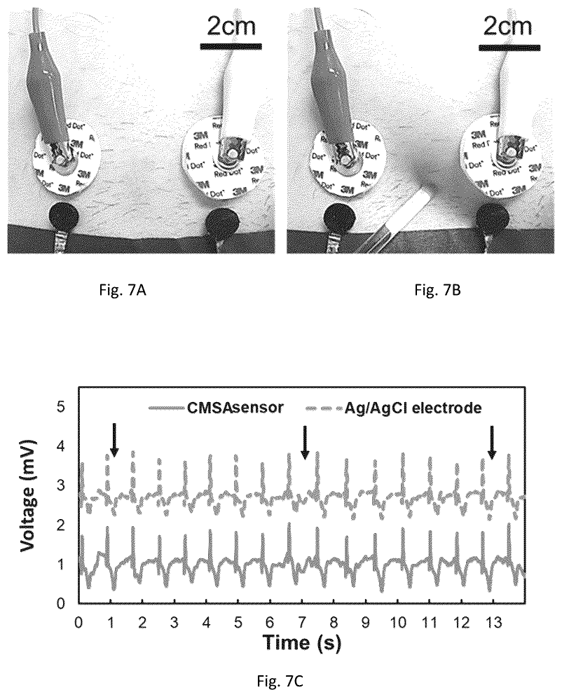

To study the susceptibility of the CMSA sensor to motion, the CMSA sensor and Ag/AgCl wet gel electrode as used above were placed about 3 cm apart on a subject's chest (FIG. 7A) and simultaneous ECG recordings were performed while motion was induced by poking skin in the vicinity of both sensors using a glass rod (FIG. 7B). A 60 Hz notch filter and a high pass filter of 1 Hz were applied to the recorded signal using MATLAB software. The results indicated that the CMSA sensor shows comparable motion artifacts to the wet gel Ag/AgCl electrode (FIG. 7C).

For long-term use, wearable sensors must be comfortable and non-irritating. To investigate this, a CMSA sensor embodiment and an Ag/AgCl wet gel electrode were placed on a subject's forearm and any signs of skin irritation 6 hours after placement were noted. Results show that the CMSA sensor did not cause any irritation and discomfort after 6 hours, and this was also the result after leaving the sensor on the skin for several days. In contrast, the medical grade Ag/AgCl 3M electrode caused irritation after 6 hours of wearing. Furthermore, the small-size, light weight, and simple application of the CMSA sensor made it comfortable to wear and easy to use.

All cited publications are incorporated herein by reference in their entirety.

### EQUIVALENTS

It will be appreciated that modifications may be made to the embodiments described herein without departing from the scope of the invention. Accordingly, the invention should not be limited by the specific embodiments set forth but should be given the broadest interpretation consistent with the teachings of the description as a whole.

### REFERENCES

[1] Baillet, S. (2017). Magnetoencephalography for brain electrophysiology and imaging. Nat Neurosci, 20(3), 327-339. https://doi.org/10.1038/nn.4504
[2] Hennig, J., Speck, O., Koch, M. A., & Weiller, C. (2003). Functional magnetic resonance imaging: a review of methodological aspects and clinical applications. J Magn Reson Imaging, 18 (1), 1-15. https://doi.org/10.1002/jmri.10330
[3] Biasiucci, A., Franceschiello, B., & Murray, M. M. (2019). Electroencephalography. Curr Biol, 29 (3), R80-R85. https://doi.org/10.1016/j.cub.2018.11.052
[4] Pillai, J., & Sperling, M. R. (2006). Interictal EEG and the diagnosis of epilepsy. Epilepsia, 47 Suppl 1, 14-22. https://doi.org/10.1111/j.1528-1167.2006.00654.x
[5] Schultz, T. L. (2012). Technical tips: MRI compatible EEG electrodes: advantages, disadvantages, and financial feasibility in a clinical setting. Neurodiagn J, 52(1), 69-81. https://www.ncbi.nlm.nih.gov/pubmed/22558648
[6] Kabiri Ameri, S., Ho, R., Jang, H., Tao, L., Wang, Y., Wang, L., Schnyer, D. M., Akinwande, D., & Lu, N. (2017). Graphene Electronic Tattoo Sensors. ACS Nano, 11(8), 7634-7641. https://doi.org/10.1021/acsnano.7b02182
[7] Leleux, P., Badier, J. M., Rivnay, J., Benar, C., Herve, T., Chauvel, P., & Malliaras, G. G. (2014). Conducting polymer electrodes for electroencephalography. Adv Healthc Mater, 3 (4), 490-493.

https://doi.org/10.1002/adhm.201300311
[8] Kim, D.-H., Lu, N., Ma, R., Kim, Y.-S., Kim, R.-H., Wang, S., Wu, J., Won, S. M., Tao, H., & Islam, A. (2011). Epidermal electronics. science, 333(6044), 838-843.
[9] Wang, L., Qiao, S., Kabiri Ameri, S., Jeong, H., & Lu, N. (2017). A thin elastic membrane conformed to a soft and rough substrate subjected to stretching/compression. Journal of Applied Mechanics, 84(11).
[10] Wang, Y., Qiu, Y., Ameri, S. K., Jang, H., Dai, Z., Huang, Y., & Lu, N. (2018). Low-cost, μm-thick, tape-free electronic tattoo sensors with minimized motion and sweat artifacts. NPJ Flexible Electronics, 2(1), 1-7.
[11] Ferrari, L. M., Ismailov, U., Badier, J.-M., Greco, F., & Ismailova, E. (2020). Conducting polymer tattoo electrodes in clinical electro- and magneto-encephalography. NPJ Flexible Electronics, 4 (1), 1-9.
[12] Xu, C., Yang, Y., & Gao, W. (2020). Skin-interfaced sensors in digital medicine: from materials to applications. Matter, 2(6), 1414-1445.
[13] Kireev, D., Ameri, S. K., Nederveld, A., Kampfe, J., Jang, H., Lu, N., & Akinwande, D. (2021). Fabrication, characterization and applications of graphene electronic tattoos. Nature Protocols, 16 (5), 2395-2417.
[14] Liu, B., Tang, H., Luo, Z., Zhang, W., Tu, Q., & Jin, X. (2017). Wearable carbon nanotubes-based polymer electrodes for ambulatory electrocardiogrameasurements. Sensors and Actuators A: Physical, 265, 79-85.
[15] Jung, J., Shin, S., & Kim, Y. T. (2019). Dry electrode made from carbon nanotubes for continuous recording of bio-signals. Microelectronic Engineering, 203, 25-30.
[16] Sun, B., McCay, R. N., Goswami, S., Xu, Y., Zhang, C., Ling, Y., Lin, J., & Yan, Z. (2018). Gas-permeable, multifunctional on-skin electronics based on laser-induced porous graphene and sugar-templated elastomer sponges. Advanced Materials, 30(50), 1804327.
[17] Liu, L., Li, H. Y., Fan, Y. J., Chen, Y. H., Kuang, S. Y., Li, Z. B., Wang, Z. L., & Zhu, G. (2019). Nanofiber-reinforced silver nanowires network as a robust, ultrathin, and conformable epidermal electrode for ambulatory monitoring of physiological signals. Small, 15(22), 1900755.
[18] Jiang, Z., Nayeem, M. O. G., Fukuda, K., Ding, S., Jin, H., Yokota, T., Inoue, D., Hashizume, D., & Someya, T. (2019). Highly stretchable metallic nanowire networks reinforced by the underlying randomly distributed elastic polymer nanofibers via interfacial adhesion improvement. Advanced Materials, 31(37), 1903446.
[19] Guo, W., Zheng, P., Huang, X., Zhuo, H., Wu, Y., Yin, Z., Li, Z., & Wu, H. (2019). Matrix-independent highly conductive composites for electrodes and interconnects in stretchable electronics. ACS applied materials & interfaces, 11(8), 8567-8575.
[20] Lee, S. M., Byeon, H. J., Lee, J. H., Baek, D. H., Lee, K. H., Hong, J. S., & Lee, S.-H. (2014). Self-adhesive epidermal carbon nanotube electronics for tether-free long-term continuous recording of biosignals. Scientific reports, 4(1), 1-9.
[21] Greiner, C., Campo, A. D., & Arzt, E. (2007). Adhesion of bioinspired micropatterned surfaces: effects of pillar radius, aspect ratio, and preload. Langmuir, 23(7), 3495-3502. https://doi.org/10.1021/la0633987
[22] Stauffer, F., Thielen, M., Sauter, C., Chardonnens, S., Bachmann, S., Tybrandt, K., Peters, C., Hierold, C., & Vörös, J. (2018). Skin conformal polymer electrodes for clinical ECG and EEG recordings. Advanced healthcare materials, 7(7), 1700994.
[23] Bae, W. G., Kim, D., Kwak, M. K., Ha, L., Kang, S. M., & Suh, K. Y. (2013). Enhanced skin adhesive patch with modulus-tunable composite micropillars. Adv Healthc Mater, 2(1), 109-113. https://doi.org/10.1002/adhm.201200098
[24] Seong, M., Hwang, I., Lee, J., & Jeong, H. E. (2020). A Pressure-Insensitive Self-Attachable Flexible Strain Sensor with Bioinspired Adhesive and Active CNT Layers. Sensors (Basel), 20(23). https://doi.org/10.3390/s20236965
[25] Kim, T., Park, J., Sohn, J., Cho, D., & Jeon, S. (2016). Bioinspired, Highly Stretchable, and Conductive Dry Adhesives Based on 1D-2D Hybrid Carbon Nanocomposites for All-in-One ECG Electrodes. ACS Nano, 10(4), 4770-4778. https://doi.org/10.1021/acsnano.6b01355
[26] Min, H., Jang, S., Kim, D. W., Kim, J., Baik, S., Chun, S., & Pang, C. (2020). Highly air/water-permeable hierarchical mesh architectures for stretchable underwater electronic skin patches. ACS applied materials & interfaces, 12(12), 14425-14432.
[27] Chun, S., Son, W., Kim, D. W., Lee, J., Min, H., Jung, H., Kwon, D., Kim, A.-H., Kim, Y.-J., & Lim, S. K. (2019). Water-resistant and skin-adhesive wearable electronics using graphene fabric sensor with octopus-inspired microsuckers. ACS applied materials & interfaces, 11(18), 16951-16957.
[28] Baik, S., Lee, J., Jeon, E. J., Park, B.-y., Kim, D. W., Song, J. H., Lee, H. J., Han, S. Y., Cho, S.-W., & Pang, C. (2021). Diving beetle-like miniaturized plungers with reversible, rapid biofluid capturing for machine learning-based care of skin disease. Science Advances, 7(25), eabf5695.
[29] Kawada, T., Kiryu, Y., Aoki, S., & Suzuki, S. (1992). Validity of electrode placement at fpz to detect alpha wave. Psychiatry and Clinical Neurosciences, 46(4), 937-940.
[30] Olejarczyk, E., Bogucki, P., & Sobieszek, A. (2017). The EEG split alpha peak: phenomenological origins and methodological aspects of detection and evaluation. Frontiers in Neuroscience, 11, 506.

1. A method for making a stemmed conical head microstructure sensor, comprising:
providing a depot of an electrically conductive polymer precursor with a selected viscosity on a substrate;
providing a mold comprising a semi-spherical structure for the conical head of the microstructure;
placing the mold above the substrate wherein the mold contacts the polymer precursor and a selected portion of the mold is wetted by the polymer precursor;
separating the mold and the substrate by a selected distance to allow the polymer precursor to be drawn between the mold and the depot while adhering to the mold according to its selected viscosity, wherein the polymer precursor drawn between the mold and the depot of the polymer precursor forms a stem of the stemmed conical head microstructure sensor;
maintaining the selected distance between the mold and the substrate while subjecting the mold and the substrate to a treatment that cures the polymer precursor to form the stemmed conical head microstructure; and
removing the conical head from the mold to obtain the stemmed conical head microstructure sensor on the substrate.

2. The method of claim 1, wherein the selected viscosity is about 2000 Pas to about 13500 P a s.

3. The method of claim 1, wherein the selected distance is about 450 μm to about 650 μm.

4. The method of claim 1, wherein the polymer precursor comprises at least one of silicone-based elastomers, polytetrafluoroethylene (PTFE), thermoplastic elastomer (TPE) family of polymers, polyurethane plastics, thermoplastic polyurethane (TPU).

5. The method of claim 1, wherein the polymer precursor comprises a filler selected from graphene, carbon nanotubes, metallic nanoparticles, carbon black, metal flakes, metal nanowire, non-metallic nanoparticles, and combinations thereof.

6. The method of claim 1, wherein the polymer precursor comprises carbon nanotube-polydimethylsiloxane-silicone oil (CNT-PDMS-SO).

7. The method of claim 6, comprising preparing the CNT-PDMS-SO precursor with the selected viscosity by dispersing CNT in isopropanol alcohol (IPA), SO, and PDMS using ultrasonication, and then heating to remove the IPA to obtain the CNT-PDMS-SO solution.

8. The method of claim 6, comprising preparing the CNT-PDMS-SO solution with the selected viscosity by mixing about 2.4 wt % CNT and about 20 wt % silicone oil in PDMS, with CNT:SO weight ratio of about 1:8.

9. The method of claim 6, wherein the CNT comprises carbon nanotubes about 10 nm to about 20 nm in diameter and about 10 μm to about 30 μm in length.

10. The method of claim 1, wherein the mold comprises one or more glass bead fixed on a face of a second substrate.

11. The method of claim 8, wherein the semi-spherical structure has a radius of about 750 μm.

12. The method of claim 1, comprising two or more semi-spherical structures wherein spacing between the semi-spherical structures is about 350 μm to about 450 μm.

13. The method of claim 1, comprising separating the mold and the substrate by the selected distance wherein the semi-spherical structure contacts the polymer precursor and about 10% to about 35% of a radius of the semi-spherical structure is wetted by the polymer precursor.

14. A sensor for measuring and/or monitoring one or more bioelectrical signals from the skin and/or applying one or more electrical signals to the skin of a subject, comprising:
a substrate;
a stemmed conical head microstructure disposed on the substrate;
wherein the stemmed conical head microstructure comprises a polymer composite having a Young's modulus of about 200 kPa to about 1.8 MPa.

15. The sensor of claim 14, wherein the Young's modulus is about 1.7 MPa.

16. The sensor of claim 14, wherein the polymer composite comprises CNT-PDMS-SO.

17. The sensor of claim 16, wherein the polymer composite comprises about 2.3 to about 2.5 wt % of CNT and about 18 to about 22 wt % of SO.

18. The sensor of claim 16, wherein the polymer composite comprises CNT-PDMS-SO with CNT:SO weight ratio of 1:8.

19. A method for measuring and/or monitoring one or more bioelectrical signals from the skin and/or applying one or more electrical signals to the skin of a subject, comprising adhering the sensor of claim 14 to the skin of the subject and measuring and/or monitoring the one or more bioelectrical signals and/or applying the one or more electrical signals.

20. The method of claim 19, wherein the one or more bioelectrical signal is selected from electromyogram (EMG), electrocardiogram (ECG), electroencephalogram (EEG), electrooculogram (EOG), electroneurogram (ENG), electrochemical skin conductance (ESC), and electrical impedance myography (EIM).

21. The method of claim 19, comprising adhering the sensor to the skin regardless of an amount or density of hair coverage on the skin.
